(19) United States
(12) Patent Application Publication
(10) Pub. No.: US 2015/0005856 A1
(43) Pub. Date: Jan. 1, 2015

(54) LEAD ANCHORS AND SYSTEMS AND METHODS USING THE LEAD ANCHORS

(71) Applicant: BOSTON SCIENTIFIC NEUROMODULATION CORPORATION, Valencia, CA (US)

(72) Inventors: Anne Margaret Pianca, Santa Monica, CA (US); Rafael Carbunaru, Valley Village, CA (US); David Ernest Wechter, Santa Clarita, CA (US)

(21) Appl. No.: 14/312,194

(22) Filed: Jun. 23, 2014

Related U.S. Application Data
(60) Provisional application No. 61/840,251, filed on Jun. 27, 2013.

(51) Int. Cl. A61N 1/05

(52) U.S. Cl. CPC A61N 1/05
USPC 607/116

(57) ABSTRACT

A lead anchor includes a body defining a lead lumen extending from a first end to a second end of the body to receive a lead, and a transverse lumen extending from a top end of the body and perpendicularly intersecting the lead lumen. Further, anchor includes a fastener disposed in the transverse lumen and configured for fastening the lead to the anchor by engaging a portion of the lead. The fastener includes a head defining an indented pattern configured to receive a tip portion of a tool. The pattern is tilted so that the tip portion of the tool can be inserted into the pattern at an angle with respect to the transverse lumen. The angle lies between 20-85 degrees as measured between a tool's longitudinal axis and a plane that contains the lead lumen's longitudinal axis and is perpendicular to the transverse lumen.

### CROSS-REFERENCE TO RELATED APPLICATIONS

This application claims the benefit under 35 U.S.C. §119(e) of U.S. Provisional Patent Application Ser. No. 61/840,251, filed Jun. 27, 2013, which is incorporated herein by reference.

### FIELD

The present invention is directed to the area of implantable electrical stimulation systems and methods of making and using the systems. The present invention is also directed to lead anchors for implantable electrical stimulation leads, as well as methods of making and using the lead anchors and electrical stimulation systems.

### BACKGROUND

Implantable electrical stimulation systems have proven therapeutic in a variety of diseases and disorders. For example, spinal cord stimulation systems have been used as a therapeutic modality for the treatment of chronic pain syndromes. Peripheral nerve stimulation has been used to treat chronic pain syndrome and incontinence, with a number of other applications under investigation. Functional electrical stimulation systems have been applied to restore some functionality to paralyzed extremities in spinal cord injury patients.

Stimulators have been developed to provide therapy for a variety of treatments. A stimulator can include a control module (with a pulse generator), one or more leads, and an array of stimulator electrodes on each lead. The stimulator electrodes are in contact with or near the nerves, muscles, or other tissue to be stimulated. The pulse generator in the control module generates electrical pulses that are delivered by the electrodes to body tissue.

### BRIEF SUMMARY

One embodiment is a lead anchor having a body that defines an outer surface, a top end, a front side, a first end, and a second end that is opposite to the first end. A longitudinal lead lumen extends within the body from the first end to the second end, and receives a lead. The body also defines a transverse lumen, which extends from the top end and perpendicularly intersects the lead lumen. The lead anchor also includes a fastener, which is disposed in the transverse lumen, for fastening the received lead to the lead anchor by engaging a portion of the lead. The fastener includes a head which defines an indented pattern for receiving a tip portion of a tool so that the tool can be used to rotate the fastener within the transverse lumen. The indented pattern is tilted with respect to the head so that the tip portion of the tool can be inserted into the indented pattern at an angle relative in the transverse lumen. In some embodiments, this angle is in a range from 20 to 85 degrees as measured between a longitudinal axis of the tool and a plane that contains the longitudinal axis of the lead lumen and that is perpendicular to the transverse lumen.

Another embodiment is a kit having an implantable stimulation lead and a lead anchor described above. The lead anchor is configured and arranged to receive a portion of the implantable stimulation lead within the lumen of the lead anchor.

Yet another embodiment is a lead anchor having a body that defines an outer surface, a front side, a first end, and a second end that is opposite to the first end. A longitudinal lead lumen extends within the body from the first end to the second end, and receives a lead. The body also defines a first transverse lumen which extends from the outer surface and perpendicularly intersects the lead lumen. The body also defines a second transverse lumen, which extends from the outer surface and perpendicularly intersects the lead lumen. The first and the second transverse lumens form an angle therebetween which is in a range from 30 to 165 degrees. The lead anchor also includes a first fastener that is disposed in the first transverse lumen and a second fastener that is disposed in the second transverse lumen. The first and the second fasteners fasten the received lead to the lead anchor by engaging a portion of the lead.

A further embodiment is a lead anchor including a body having an outer surface, a top end, a front side, a first end, and a second end opposite to the first end. The body defines a lead lumen to receive a lead, the lead lumen defining an elongated opening extending along the front aide of the body from the first end to the second end. The body further defines a transverse lumen extending from the top end of the body and intersecting the lead slot. The lead anchor also includes a first strain relief arrangement extending along a portion of the body adjacent the first end of the body. The first strain relief portion includes an alternating series of first concentric elements with differing outer diameter, where the first concentric elements facilitate axial deformation of the first end of the body. The lead anchor further includes a second strain relief arrangement extending along a portion of the body adjacent the second end of the body. The second strain relief portion includes an alternating series of second concentric elements with differing enter diameters, where the second concentric elements facilitate axial deformation of the second end of the body. The lead anchor also includes a fastener disposed in the transverse lumen for fastening the received lead to the lead anchor by engaging a portion of the lead.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic side view of one embodiment of an electrical stimulation system that includes a paddle lead electrically coupled to a control module, according to the invention;

FIG. 2 is a schematic side view of one embodiment of an electrical stimulation system that includes a percutaneous lead electrically coupled to a control module, according to the invention;

FIG. 3A is a schematic side view of one embodiment of the control module of FIG. 1 configured and arranged to electrically couple to an elongated device, according to the invention;

FIG. 3B is a schematic side view of one embodiment of a lead extension configured and arranged to electrically couple the elongated device of FIG. 2 to the control module of FIG. 1, according to the invention;

FIG. 4 is a schematic perspective side view of one embodiment of a lead anchor and a tool, according to the invention;

FIG. 5 is a schematic cross-sectional view of one embodiment of a lead anchor, according to the invention;

FIG. 6 is a schematic perspective side view of a second embodiment of a lead anchor and a tool, according to the invention;

FIG. 7 is a schematic perspective side view of a third embodiment of a lead anchor, according to the invention;

FIG. 8 is a schematic perspective side view of a fourth embodiment of a lead anchor, according to the invention;

FIG. 9 is a schematic tip view of a fifth embodiment of a lead anchor, according to the invention;

FIG. 10 is a schematic overview of one embodiment of components of a stimulation system, including an electronic subassembly disposed within a control module, according to the invention.

### DETAILED DESCRIPTION

Some embodiments are directed to implantable electrical stimulation systems and methods of making and using the systems. Some of these embodiments are particularly directed to lead anchors for implantable electrical stimulation leads having a set screw locking mechanism, as well as methods of making and using the lead anchors and electrical stimulation systems.

Suitable implantable electrical stimulation systems include, but are not limited to, a least one lead with one or more electrodes disposed along a distal end of the lead and one or more terminals disposed along the one or more proximal ends of the lead. Leads include, for example, percutaneous leads, paddle leads, and cuff leads. Examples of electrical stimulation systems with leads are found in, for example, U.S. Pat. Nos. 6,181,969; 6,516,227; 6,609,029; 6,609,032; 6,741,892; 7,949,395; 7,244,150; 7,672,734; 7,761,165; 7,974,706; 8,175,710; 8,224,450; and 8,364,278; and U.S. Patent Application Publication No. 2007/0150036, all of which are incorporated by reference.

Some embodiments provide a lead anchor that is beneficial, as compared to a conventional lead anchor, by making it easier to engage the lead anchor's fastener (e.g., a set screw). For example, limited space is often available to engage the lead anchor's fastener (e.g., set screw) during surgical operations, particularly those involving small incisions.

In at least some conventional lead anchors, the fastener mechanism can only be engaged when a tool (such as a hex wrench) is disposed at an approximately 90 degree angle relative to the main axis of a lead. A novel lead anchor can be designed so that the fastener mechanism can be engaged when the tool is at an angle that is greater than zero degrees and less than 90 degrees relative to the main axis of the lead.

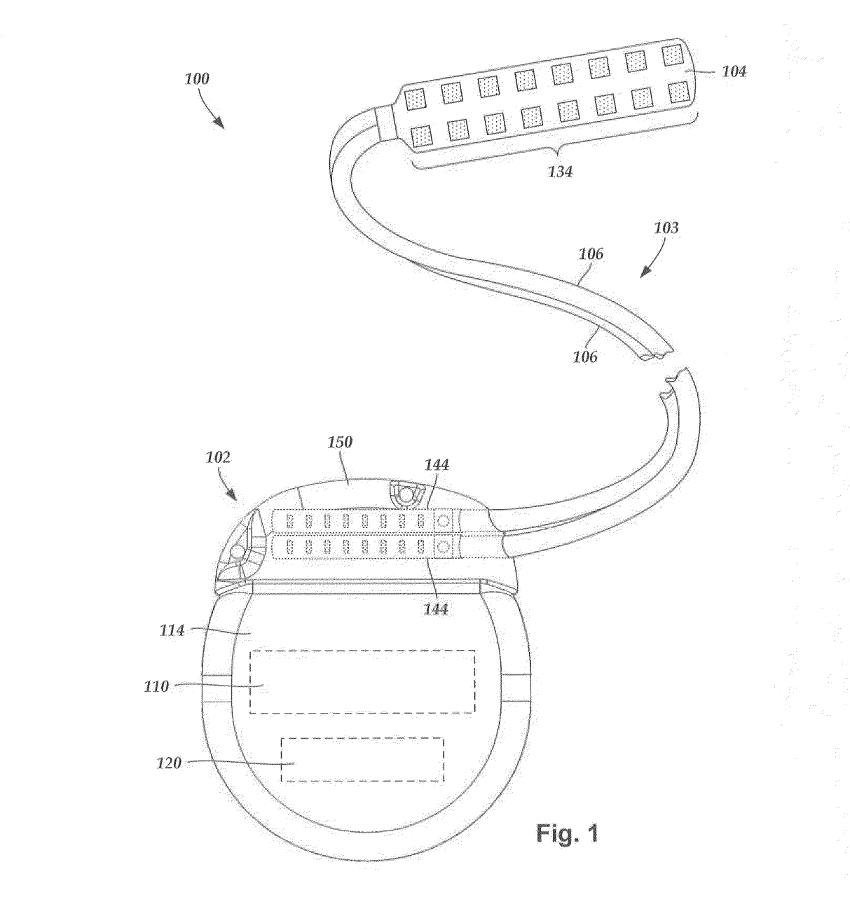

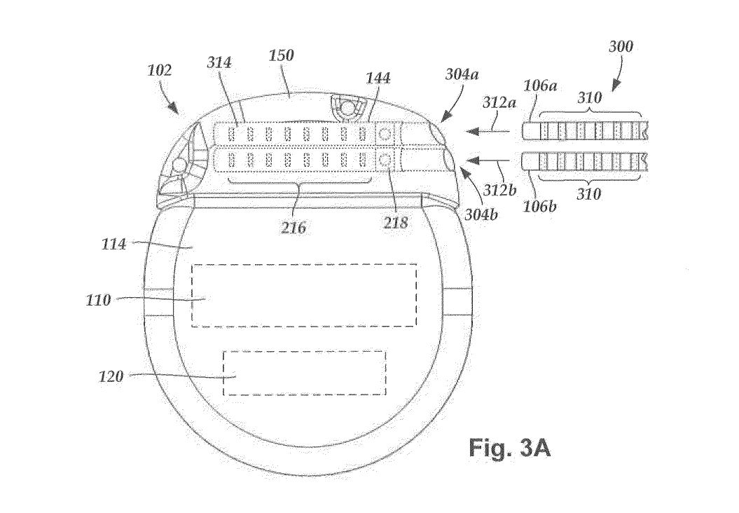

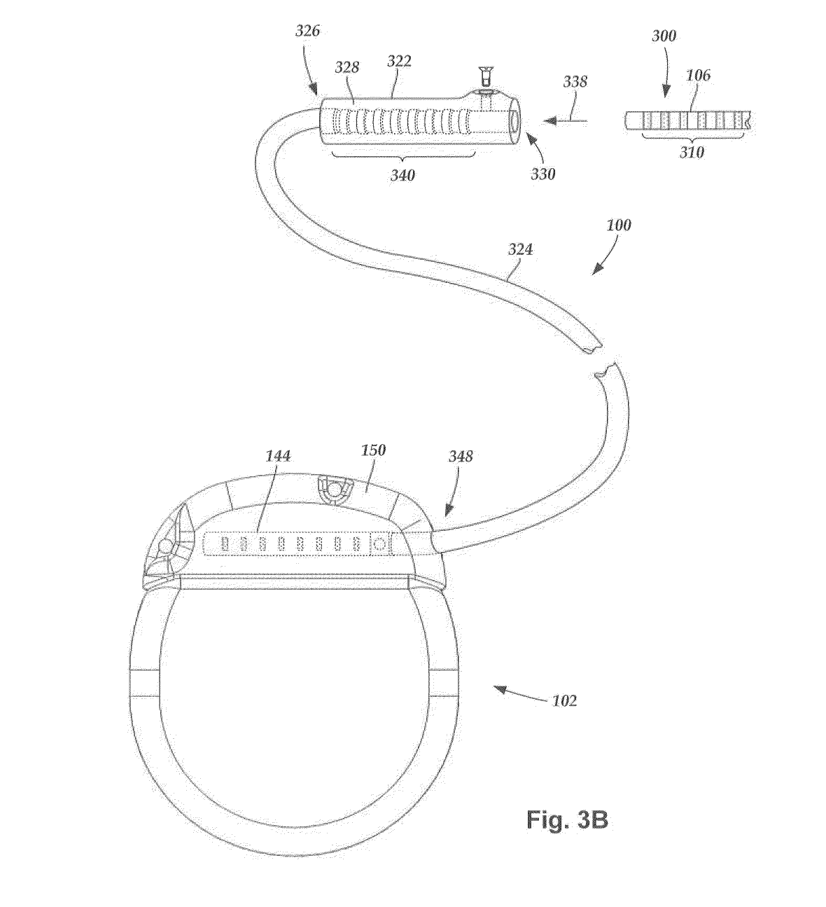

FIG. 1 schematically illustrates an electrical stimulation system 100 according to one embodiment of the invention. The electrical stimulation system 100 includes a control module (e.g., a stimulator or pulse generator) 102 and a lead 103 that can be coupled or otherwise connected to the control module 102. The lead 103 includes a paddle body 104 and one or more lead bodies 106. The lead 103 of the embodiment shown in FIG. 1 has two lead bodies 106. However, the lead 103 can include any suitable number of lead bodies including, for example, one, two, three, four, five, six, seven, eight or more lead bodies 106. An array of electrodes 133, including multiple individual electrodes 134, is disposed on the paddle body 104, and an array of terminals (e.g., indicated by reference number 310 in FIGS. 3A and 3B) is disposed along each of the one or more lead bodies 106, such as at the end opposite the paddle body 104.

The above structures are merely provided for exemplary purposes, and the electrical stimulation system can include more, fewer, or different components than the components disclosed above. In fact, the electrical stimulation system can have a variety of different configurations including the electrical stimulation system configurations disclosed in the documents incorporated herein by reference. For example, instead, of a paddle body, the electrodes can be disposed in an array at or near the distal end of a lead body forming a percutaneous lead.

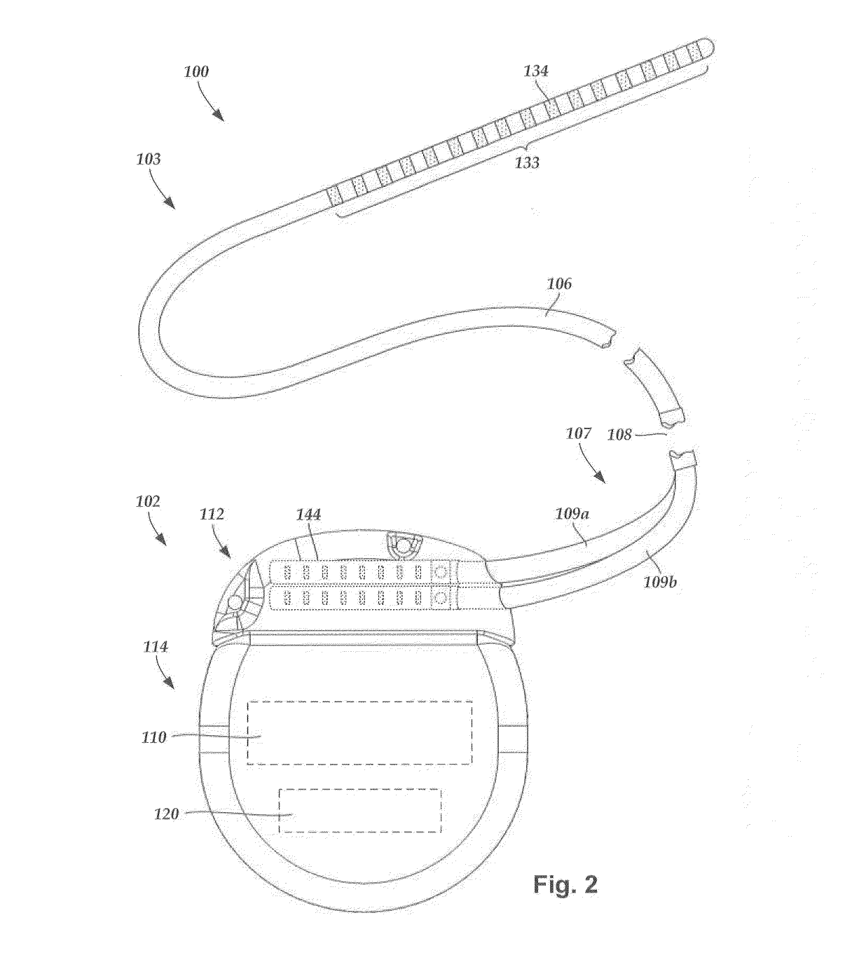

FIG. 2 schematically illustrates an electrical stimulation system 100 according to another embodiment, where the lead 103 is a percutaneous lead. The electrodes 134 are shown in FIG. 2 as being disposed linearly along the one or more lead bodies 106. In at least some embodiments, the lead 103 is isodiametric (or has a uniform or substantially uniform diameter) along a longitudinal length of the lead body 106. The electrodes 134 can be uniformly spaced along the lead body 106 as shown in FIG. 2 or alternatively spaced at irregular intervals.

The lead 103 can be coupled to the control module 102 in any suitable manner. The lead 103 is shown in FIG. 1 as being coupled directly to the control module 102. However, the lead 103 of at least some other embodiments is coupled indirectly to the control module 102, such as by being coupled to the control module 102 via one or more intermediate devices (indicated by reference number 300 in FIGS. 3A-3B). For example, in at least some embodiments, one or more lead extensions 324 (see e.g., FIG. 3B) can be disposed between the lead 103 and the control module 102 to extend the distance between the lead 103 and the control module 102. Other intermediate devices may be used in addition to, or in lieu of, the one or more lead extensions 324 including, for example, a splitter, an adaptor, combinations thereof, and/or any other known, related art or later developed apparatus. In addition, in configurations where the electrical stimulation system 100 includes multiple elongated devices disposed between the lead 103 and the control module 102, the intermediate devices may be configured into any suitable arrangement.

The electrical stimulation system 100 is shown in FIG. 2 as having a splitter 107 for facilitating coupling of the lead 103 to the control module 102. The splitter 107 includes a splitter connector 108 configured to couple to a proximal end of the lead 103, and one or more splitter tails 109a and 109b configured and arranged to couple to the control module 102 (or another splitter, lead extension, adaptor, or the like).

The control module 102 can include a connector housing 112 and a sealed electronics housing 114. An electronic subassembly 110 and an optional power source 120 are disposed in the electronics housing 114. A control module connector 144 is disposed in the connector housing 112. The control module connector 144 is configured and arranged to make an electrical connection between the lead 103 and the electronic subassembly 110 of the control module 102.

The electrical stimulation system or components thereof, including the paddle body 104, the one or more of the lead bodies 106, and the control module 102, can be implanted into a patient's body. The electrical stimulation system can be used for a variety of applications including, but not limited to, deep brain stimulation, neural stimulation, spinal cord stimulation, muscle stimulation, and the like.

The electrodes 134 can be formed using any conductive, biocompatible material. Examples of suitable materials include metals, alloys, conductive polymers, conductive carbon, and the like, as well as combinations thereof. In at least some embodiments, one or more of the electrodes 134 are formed from one or more of: platinum, platinum iridium, palladium, palladium rhodium, or titanium.

Any suitable number of electrodes 134 can be disposed on the lead 103 including, for example, four, five, six, seven, eight, nine, ten, eleven, twelve, fourteen, sixteen, twenty-four, thirty-two, or more electrodes 134. In the case of paddle leads, the electrodes 134 can be disposed on the paddle body 104 in any suitable arrangement. The electrodes 134 are shown in FIG. 1 arranged into two columns, where each column has eight electrodes 134.

The electrodes of the paddle body 104 (or one or more lead bodies 106) can be disposed in, or separated by, a non-conductive, biocompatible material, such as, for example, silicone, polyurethane, polyetheretherketone (“PEEK”), epoxy, and the like, or combinations thereof. The one or more lead bodies 106 and, if applicable, the paddle body 104, may be formed into any desired shape by any suitable process including, for example, molding (including injection molding), casting, and the like. The non-conductive material of the one or more lead bodies 106 can extend from its distal end to its proximal end.

In the case of paddle leads, the non-conductive material typically extends from the paddle body 104 to the proximal end of each of the one or more lead bodies 106. Additionally, the non-conductive, biocompatible material of the paddle body 104 can be the same material as the one or more lead bodies 106 or alternatively n can be different. Moreover, the paddle body 104 and the one or more lead bodies 106 may be a unitary structure or can be formed as two separate structures that are permanently or detachable coupled together.

Terminals (e.g., indicated by reference number 310 in FIGS. 3A-3B) can be disposed along the proximal end of the one or more lead bodies 106 of the electrical stimulation system 100 (as well as any splitters, lead extensions, adaptors, or the like) for electrical connection to corresponding connector contacts (e.g., indicated by reference number 314 in FIGS. 3A-3B). The connector contacts 314 are disposed in connectors or the control module connector (e.g., indicated by reference number 144 in FIGS. 1-3B; and reference number 322 FIG. 3B) which, in turn, are disposed on, for example, the control module 102 (or a lead extension, splitter, adaptor, or the like). Electrically conductive wires, cables, etc. (not shown) extend from the terminals 310 to the electrodes 134. One or more of the electrodes 134 can be electrically coupled to each terminal 310. In at least some embodiments, each terminal 310 is only connected to one electrode 134.

The electrically conductive wires (“conductors”) may be embedded in the non-conductive material of the lead body 106, or alternatively can be disposed in one or more lumens (not shown) extending along the lead body 106. In some embodiments, an individual lumen is provided for each conductor. In other embodiments, two or more conductors extend through a single lumen. One or more lumens (not shown) may be provided that open at, or near, the proximal end of the one or more lead bodies 106. A stylet may be inserted into these lumens to facilitate placement of the one or more lead bodies 106 within a patient's body. Additionally, one or more lumens (not shown) may be provided that open at, or near, the distal end of the one or more lead bodies 106 for infusion of drugs or medication into the site of implantation of the one or more lead bodies 106. In at least one embodiment, the one or more lumens are flushed continually, or on a regular basis, with saline, epidural fluid, or the like. In at least some embodiments, the one or more lumens are permanently or removably sealable at the distal end of the one or more lead bodies 106.

FIG. 3A is a schematic side view of one embodiment of a proximal end of one or more elongated devices 300 configured and arranged for coupling to one embodiment of the control module connector 144. The one or more elongated devices may include, for example, the one or more of the lead bodies 106 of FIG. 1, one or more intermediate devices (e.g., a splitter, the lead extension 324 of FIG. 3B, an adaptor, or the like or combinations thereof), or a combination thereof.

The control module connector 144 defines at least one port into which a proximal end of the elongated device 300 can be inserted, as shown by directional arrows 312a and 312b. In FIG. 3A (and in other figures), the connector housing 112 is shown as having two ports 304a and 304b. The connector housing 112 can define any suitable number of ports including, for example, one, two, three, four, five, six, seven, eight, or more ports.

The control module connector 144 also includes a plurality of connector contacts, such as connector contact 314, disposed within each port 304a and 304b. When the elongated, device 300 is inserted into the ports 304a and 304b, the connector contacts 314 can be aligned with a plurality of terminals 310 disposed along the proximal end(s) of the elongated devices(s) 300 to electrically couple the control module 102 to the electrodes (134 of FIG. 1) disposed on the paddle body 104 of the lead 103. Embodiments are intended to include or otherwise cover any type of connectors for use with the control modules, including but not limited to those disclosed in U.S. Pat. Nos. 7,244,150 and 8,224,450, which are incorporated by reference.

FIG. 3B is a schematic side view of another embodiment of the electrical stimulation system 100. The electrical stimulation system 100 includes a lead extension 324 that is configured and arranged to couple one or more elongated devices 300 (e.g., one of the lead bodies 106 of FIGS. 1 and 2, the splitter 107 of FIG. 2, an adaptor, another lead extension, or the like or combinations thereof) to the control module 102. In FIG. 3B, the lead extension 324 is shown coupled to a single port 304 defined in the control module connector 144. Additionally, the lead extension 324 is shown configured and arranged to couple to a single elongated device 300. In alternate embodiments, the lead extension 324 is configured and arranged to couple to multiple ports 304 defined in the control module connector 144, or to receive multiple elongated devices 300, or both.

A lead extension connector 322 is disposed on the lead extension 324. In FIG. 3B, the lead extension connector 322 is shown disposed at a distal end 326 of the lead extension 324. The lead extension connector 322 includes a connector housing 328. The connector housing 328 defines at least one port 330 into which terminals 310 of the elongated device 300 can be inserted, as shown by directional arrow 338. The connector housing 328 also includes a plurality of connector contacts, such as connector contact 340. When the elongated device 300 is inserted into the port 330, the connector contacts 240 disposed in the connector housing 328 can be aligned with the terminals 310 of the elongated device 300 to electrically couple the lead extension 324 to the electrodes (134 of FIGS. 1 and 2) disposed along the lead (103 in FIGS. 1 and 2).

In at least some embodiments, the proximal end of the lead extension 324 is similarly configured and arranged as a proximal end of the lead 103 (or other elongated device 300). The lead extension 324 may include a plurality of electrically conductive wires (not shown) that electrically couple the connector contacts 340 to a proximal end 348 of the lead extension 324 that is opposite to the distal end 326. In at least some embodiments, the conductive wires disposed in the lead extension 324 can be electrically coupled to a plurality of terminals (not shown) disposed along the proximal end 348 of the lead extension 324. In at least some embodiments, the proximal end 348 of the lead extension 324 is configured and arranged for insertion into a connector disposed in another lead extension (or another intermediate device). In other embodiments (and as shown in FIG. 3B), the proximal end 348 of the lead extension 324 is configured and arranged for insertion into the control module connector 144.

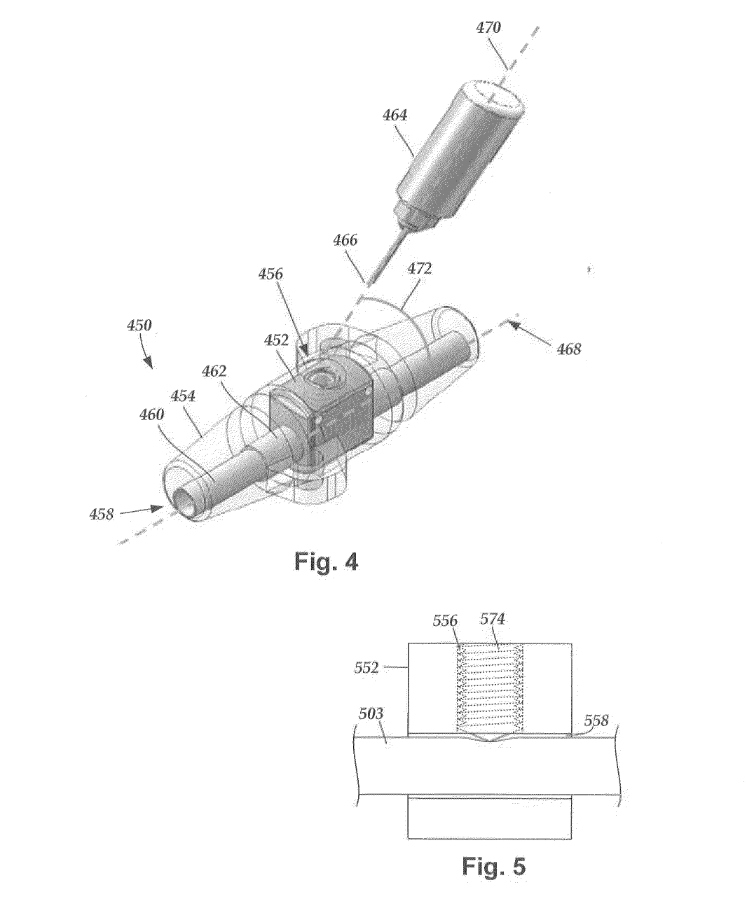

FIG. 4 is a schematic perspective side view of one embodiment of a lead anchor 450 and a tool 464. As shown, the lead anchor 450 includes an anchor body 452 (referred to as anchor body 552 in FIG. 5) having an outer surface, a top end, a front side, a first end, and a second end opposite to the first end. The lead anchor 450 and anchor body 452 defines a longitudinal lead lumen 458 (referred as lead lumen 558 in FIG. 5), which can extend from the first end of the lead anchor 450 and anchor body 452 to the second end of the lead anchor 450 cud anchor body 452, to receive a lead (such as lead 503 of FIG. 5). The lead lumen 458 can be an elongated tubular passageway within the lead anchor 450. The lead lumen 458 may have any suitable cross-section such as, but not limited to, rectangular, elliptical, circular, square, triangular, and so forth depending on shape, size and cross-section of the lead 503.

Further, the lead anchor 450 includes an exterior member 454 disposed around at least a portion of the anchor body 452 and other components of the lead anchor 450. The exterior member 454 may be formed using a biocompatible, flexible material. In some embodiments, a lead tube 460 may define or include the lead lumen 458. The lead tube 460 may extend from a distal end of the exterior member 454 to a proximal end of the exterior member 454. The exterior member 454 may further define a groove opening, bore, or any other suitable structure for fixing the lead anchor 450 to or with the tissue. The exterior member 454 can be formed using any suitable biocompatible material such as, but not limited to, a polyvinyl chloride, polyurethane, silicone, thermoplastic polysters, polycarbonate fluoropolymers, and so forth.

The anchor body 452 defines a transverse lumen 456 (referred to as transverse lumen 556 in FIG. 5) extending from the top end of the body 452 and intersecting (preferably, perpendicularly) the lead lumen 458. The transverse lumen 456 is configured and arranged to accept a fastener (see fastener 574 in FIG. 5). The transverse lumen 456 may have any suitable cross-section, such as, but not limited to, circular, triangular, rectangular, elliptical, and so forth, to hold and engage the fastener. In some embodiments, the transverse lumen 456 may be in the form of a cylindrical bore in the anchor body 452. The transverse lumen 456 may also include at least one of thread(s), rib(s), groove(s), channel(s), and so forth, to engage the fastener within.

The lead anchor 450 may also include a sleeve 462 disposed around a portion of the lead lumen 458 or the lead tube 460. The sleeve 462 may reduce, mitigate, or prevent damage to the lead caused by the fastener. The sleeve 462 may be a substantially tubular body or a sheath disposed to prevent contact between the fastener and the lead. The sleeve 462 may be formed using any suitable biocompatible polymer, such as, but not limited to, silicone, PVC, polyurethane, and so forth. In one embodiment, the sleeve 462 is a Pellethane™ sleeve. The lead tube 460 receives a portion of thee lead. Further, the lead tube 460 may be a tubular structure and/or have any suitable cross-section according to shape/size/cross-section of the lead or lead lumen 458. The lead tube 460 may include rides, threads, and so forth, for increasing friction or resistance between the lead and the lead tube 460 so as to enable engagement of the lead within the lead tube 460. In some embodiments, the lead tube 460 and the lead may be formed of the same or similar material, such as silicone, to facilitate or ensure connection between the lead and the lead tube 460 because of sealing property of the material (e.g., silicone). Further, the lead tube can be formed using any suitable biocompatible material such as, polymer, metals, alloys, plastics, and so forth. Examples of the suitable material can include, but not limited to, titanium, polyurethane, PVC, polycarbonates, aluminum, silicone, and so forth.

A tool 464 is used to fasten or tighten the fastener which in turn fastens the received lead to the lead anchor 450 by engaging a portion of the lead directly or indirectly through the sleeve or lead tube. While tightening the fastener, the tool 464 may remain at a particular angle with respect to the longitudinal axis of the lead lumen 458. Examples of the tool 464 can include, but are not limited to, a screwdriver, a wrench, an Allen wrench, and so forth. The angle 472 can be formed between a longitudinal axis 470 (hereinafter, tool axis 470) of the tool 464 and a plane that contains the longitudinal axis of the lead lumen 458 and is perpendicular to the transverse lumen 456. The angle 472 can be within a range from, for example, 20 to 85 degrees or 30 to 80 degrees or 35 to 75 degrees. The tool axis 470 is an imaginary line passing through the center of the tool 464.

In at least some embodiments, the tool 464 can be a torque limiting tool in which, a handle portion of the tool may still rotate once the limit is reached but the rotation is not conveyed to the fastener. The torque limiting tool (i.e. tool 464) can avoid over tightening of the fastener, and in turn may prevent the lead 503 from possible damage that may occur due to overtightening of the fastener.

In another embodiment, the tool can be a ball end hex wrench tool. The tip portion 466 of the ball end hex wrench tool may be in form of a hex ball.

FIG. 5 is a schematic cross-sectional view of one embodiment of an anchor body 552 for a lead anchor such as the lead anchor 450. The anchor body 552 also includes a fastener 574 disposed in the transverse lumen 556 for fastening the received lead 503 to the lead anchor 450 by engaging a portion of the lead 503. As discussed with reference to FIG. 4, the fastener 574 may also include at least one of groove(s), thread(s), channel(s), rib(s) corresponding to the threads, grooves, ribs, or the like of the transverse lumen 556.

The fastener 574 includes a head, which can define an indented pattern (not shown) to receive a tip portion 466 of the tool 464 so that the tool 464 can be used to rotate the fastener 574. The tool or the fastener may allow engagement of the tool with the fastener within the transverse lumen at angles less than 90 degrees. This can provide for more options when the lead anchor 450 is placed in an incision, as its common when implanting a lead and subsequent anchor 450. The material of the fastener 574 may include any suitable biocompatible material, such as, metal or metallic alloys. Examples of suitable material may include, but are not limited to, stainless steel, aluminum, platinum, silver, titanium, nickel, nitinol, and so forth. When the fastener 574 is completely tightened, then the fastener 574 may press against the sleeve 462 or lead 503 to hold the lead within the lead lumen 558.

In some embodiments, the indented pattern can be tilted with respect to the head or the fastener 574 so that the tip portion 466 of the tool 464 can be inserted into the indented pattern at the angle 472 with respect to the transverse lumen 456 (or 556). Further, the lead 503 once positioned within the lead tube 460 can be secured by fastening the lead 503 using the fastener 574 disposed within the transverse lumen 456. Thereafter, the lead anchor 450 may be secured to a tissue of the patient by connecting the exterior member 454 to the tissue using items, such as, suture(s), staples (s), or the like.

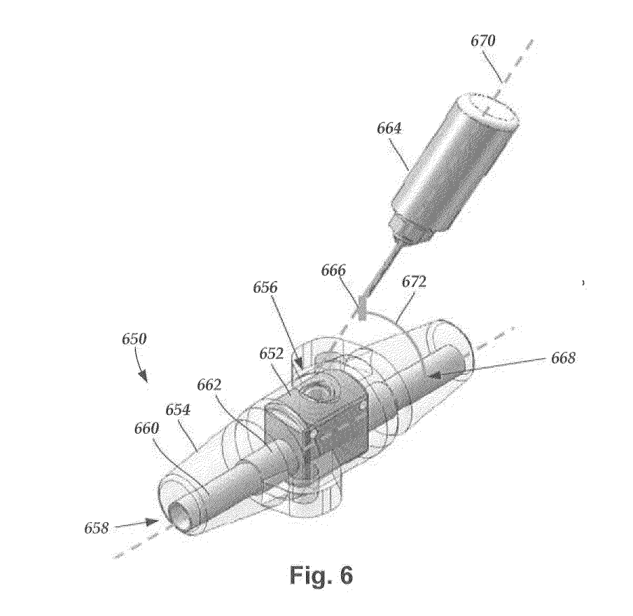

FIG. 6 is a schematic perspective side view of another embodiment of a lead anchor 650 and a tool 664. As discussed with reference to FIGS. 4 and 5, the lead anchor 650 includes a lead body 652, an exterior member 654, a transverse lumen 656, a lead lumen 658, a lead tube 660, and a sleeve 662, which are similar m functionality and structure to the anchor body 452, the exterior member 454, the transverse lumen 456, the lead lumen 458. the lead tube 460, and the sleeve 462 of the lead anchor 450, Further, the lead anchor 650 may include a fastener similar to the fastener 574.

The tool 664 is used to fasten the fastener. The tool 664 has a longitudinal axis 670. An angle 672 is formed between the tool axis 670 and a longitudinal axis 668 of the lead lumen 658. The angle 672 can be within a range from 20 to 85 degrees or 30 to 80 degrees or 35 to 75 degrees. The tool axis 670 is an imaginary line passing through the center of the tool 664. The tool 664 has a handle towards a proximal end of the tool 664 and a tip portion 666 which is tilted at an angle with respect to the tool's axis 670 and tool's handle. The tip portion 666 may engage with the head of the set screw (within the transverse lumen 556) perpendicularly. The handle of the tool 664 may rotate relative to the tip portion 666. Further, the tilted tip portion 666 may provide the flexibility to adjust the handle of the tool 664 to access a set screw at angles other than 90 degrees.

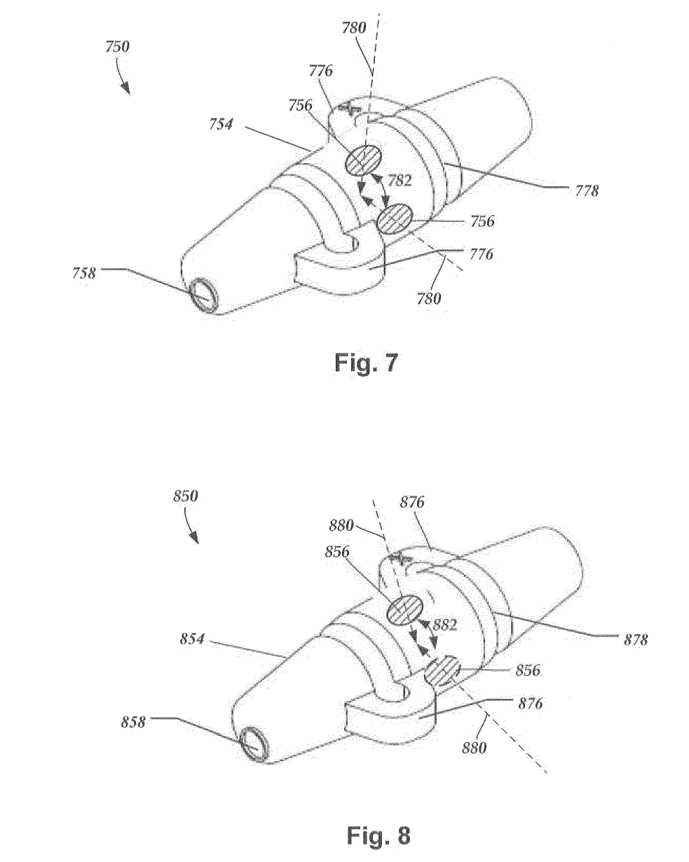

FIG. 7 is a schematic perspective side view of a third embodiment of a lead anchor 750. The lead anchor 750 is similar in functionality and structure to the lead anchor 450 of FIG. 4. The lead anchor 750 includes at least one tab 776 extending from the lead to attach the lead anchor 750 to patient's tissue. In some embodiments, an exterior member 754 defines the at least one tab 776. In some embodiments, the exterior member 754 may define a first tab and a second tab (or tabs 776) extending from an outer surface of the lead anchor 750. The second tab (tab 776) can extend in a direction opposite to the first tab (tab 776).

The lead anchor 750 may also include one or more suture grooves 778 for attaching the lead anchor 750 to the patient's tissue. The suture groove 778 can be an opening, an aperture, a groove, or the like such that the suture can pass from the suture groove while attaching the lead anchor 750 to the tissue. This secures the lead anchor 750 with the tissue. The suture may be placed in the suture grooves around the entire lead anchor 750.

In some embodiments, the lead anchor 750 may include more than one transverse lumen 756. FIG. 7 illustrates an embodiment with two transverse lumens 756. The first and second transverse lumens 756 extend from the outer surface of the anchor body 752 and may perpendicularly intersect a lead lumen 758. Further, the first and the second transverse lumens 756 may be arranged around a circumference of the lead anchor 750 such that an angle 782 is formed between the axes 780 of the first transverse lumen and the second transverse lumen. The angle 782 may range from 30 degrees to 165 degrees. Such an arrangement allows an operator to access set screw from different directions. In an embodiment, the angle 782 is within a range from 75 degrees to 100 degrees. As shown in FIG. 7, the two transverse lumens 756 may be located at 45 degree angles to the plane that contains the longitudinal axis of the lead and 90 degrees angle 782 with respect to each other.

FIG. 8 is a schematic perspective side view of a fourth embodiment of a lead anchor 850. The lead anchor 850 includes an exterior member 854, two transverse lumens 856, a lead lumen 858, at least two tabs 876, and suture grooves 878 which are functionally and structurally similar to the exterior member 754, the transverse lumens 756, the lead lumen 758, the tabs 776, and the suture grooves 778 of FIG. 7. The first and second transverse lumens 856 are located on a circumference of the lead anchor 850 at an angle 882 from each other. In FIG. 8, the first transverse lumen 856 forms an angle 882 with respect to a second transverse lumen 856, and the angle 882 is within a range of 110 to 130 degrees. In an embodiment, the angle 882 can be 120 degrees. The angle 882 is formed between transverse axes 880 of first and second transverse lumens 856.

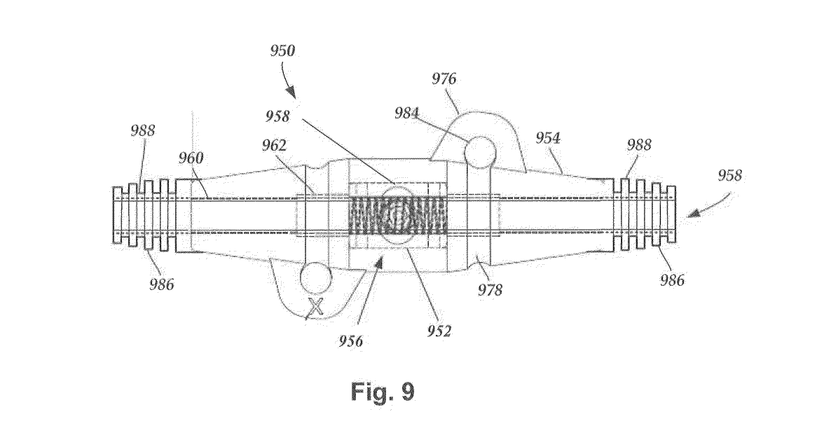

FIG. 9 is a schematic top view of a fifth embodiment of a lead anchor 950. The lead anchor 950 includes an anchor body 952 having an outer surface, a top end, a front side, a first end, and a second end opposite to the first end. The anchor body 952 defines a lead lumen 958 configured and arranged to receive a lead. The lead lumen 958 defines an elongated opening extending along the front side of the body 952 from the first end to the second end. The anchor body 952 also defines a transverse lumen 956 extending from the top end of the body 952 and intersecting the lead lumen 958.

The lead anchor also includes one or more strain relief arrangements 986 extending along the distal end or proximal end or both ends of the lead anchor. The first strain relief arrangement 986 includes an alternating series of strain relief elements 988 (for example, concentric elements) with differing outer diameters. The concentric elements 988 can facilitate axial deformation of the lead anchor. In some embodiments, the strain relief arrangement(s) 986 have a bellows-like or accordion-like structure. The strain relief elements 988 can have any suitable cross-section such as, circular, rectangular, elliptical, and the like. The alternating series of concentric elements 988 of the strain relief arrangement 986 may be arranged such that, for each concentric element, either a) all immediately adjacent concentric elements 988 have a larger outer diameter or b) all immediately adjacent concentric elements 988 have a smaller outer diameter.

The strain relief arrangements 986 at both ends of the lead anchor 950 may reduce or prevent damage to the lead. The first and second strain relief arrangements 986 may form a unitary structure with the lead anchor 950. Alternatively, the strain relief arrangements 986 may be formed separately and attached to the lead anchor by using suitable methods, such as, adhesive bonding, thermal bonding, and the like.

The lead anchor 950 also includes an exterior member 954 disposed around at least a portion of the anchor body 952. The exterior member 954, the first strain relief arrangement 986, and the second strain relief arrangement 986 can be formed of a biocompatible, flexible material. In one embodiment, lead anchor 950 has an outer housing of silicone and also the strain relief arrangement 986 or strain relief elements 988 can also be formed using similar material, such as, silicone or the like.

The lead anchor 950 also includes a lead tube 960. The lead anchor 950 also includes a fastener (not shown) disposed in a transverse lumen 956. The fastener may fasten the received stimulation lead to the lead anchor 950 by engaging a portion of the lead.

Strain relief can help reduce or mitigate lead fractures and lead migration issues. Further, incorporating the strain arrangement system 986 at each end of the anchor body 952 can allow axial deformation of the lead anchor 950 to occur. The deformation of the lead anchor 950 may take precedence over lead deformation.

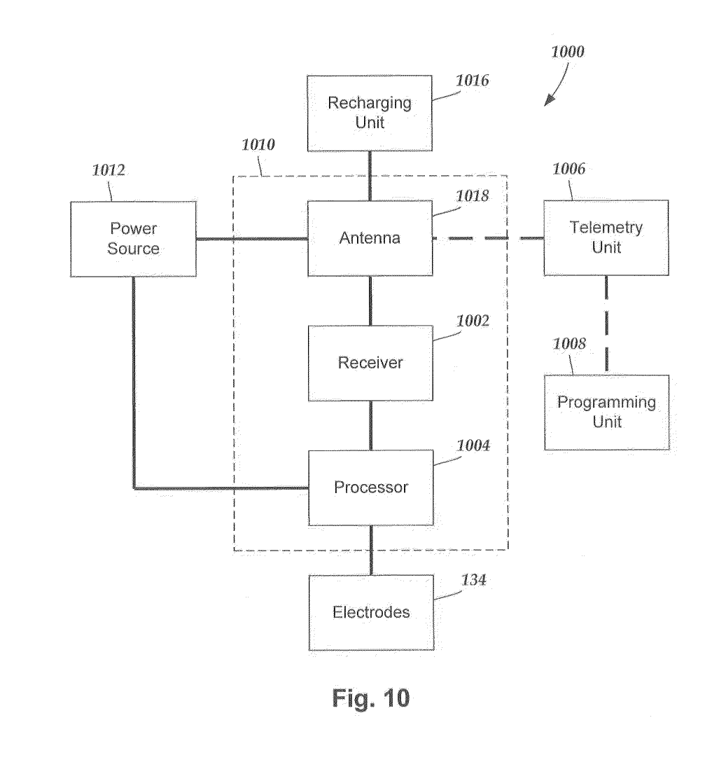

FIG. 10 is a schematic overview of one embodiment of components of an electrical stimulation system 1000 including an electronic subassembly 1010 disposed within a control module. It will be understood that the electrical stimulation system can include more, fewer, or different components and can have a variety of different configurations including those configurations disclosed in the stimulator references cited herein.

Some of the components (for example, a power source 1012, an antenna 1018, a receiver 1002, and a processor 1004) or the electrical stimulation system can be positioned on one or more circuit boards or similar carriers within a sealed housing of an implantable pulse generator, if desired. Any power source 1012 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Pat. No. 7,437,193, incorporated herein by reference.

As another alternative, power can be supplied by an external power source through inductive coupling via the optional antenna 1018 or a secondary antenna. The external power source can be in a device that is mounted on the skin of the user or in a unit that is provided near the user on a permanent or periodic basis.

If the power source 1012 is a rechargeable battery, the battery may be recharged using the optional antenna 1018, if desired. Power can be provided to the battery for recharging by inductively coupling the battery through the antenna to a recharging unit 1016 external to the user. Examples of such arrangements can be found in the references identified above.

In one embodiment, electrical current is emitted by the electrodes 134 on the paddle or lead body to stimulate nerve fibers, muscle fibers, or other body tissues near the electrical stimulation system. The processor 1004 is generally included to control the timing and electrical characteristics of the electrical stimulation system. For example, the processor 1004 can, if desired, control one or more of the timing, frequency, strength, duration, and waveform of the pulses. In addition, the processor 1004 can select which electrodes can be used to provide stimulation, if desired. In some embodiments, the processor 1004 selects which electrode(s) are cathodes and which electrode(s) are anodes. In some embodiments, the processor 1004 is used to identity which electrodes provide the most useful simulation of the desired tissue.

Any processor can be used and can be as simple as an electronic device that, for example, produces pulses at a regular interval or the processor can be capable of receiving and interpreting instructions from an external programming unit 1008 that, for example, allows modification of pulse characteristics. In the illustrated embodiment, the processor 1004 is coupled to a receiver 1002 which, in turn, is coupled to the optional antenna 1018. This allows the processor 1004 to receive instructions from an external source to, for example, direct the pulse characteristics and the selection of electrodes, if desired.

In one embodiment the antenna 1018 is capable of receiving signals (e.g., RF signals) from an external telemetry unit 1006 which is programmed by the programming unit 1008. The programming unit 1008 can be external to, or part of, the telemetry unit 1006. The telemetry unit 1006 can be a device that is worn on the skin of the user or can be carried by the user and can have a form similar to a pager, cellular phone, or remote control, if desired. As another alternative, the telemetry unit 1006 may not be worn or carried by the user but may only be available at a home station or at a clinician's office. The programming unit 1008 can be any unit that can provide information to the telemetry unit 1006 for transmission to the electrical stimulation system 1000. The programming unit 1008 can be part of the telemetry unit 1006 or can provide signals or information to the telemetry unit 1006 via a wireless or wired connection. One example of a suitable programming unit is a computer operated by the user or clinician to send signals to the telemetry unit 1006.

The signals sent to the processor 1004 via the antenna 1018 and the receiver 1002 can be used to modify or otherwise direct the operation of the electrical stimulation system. For example, the signals may be used to modify the pulses of the electrical stimulation system such as modifying one or more of pulse duration, pulse frequency, pulse waveform, and pulse strength. The signals may also direct the electrical stimulation system 1000 to cease operation, to start operation, to start charging the battery, or to stop charging the battery. In other embodiments, the stimulation system does not include the antenna 1018 or receiver 1002 and the processor 1004 operates as programmed.

The electrical stimulation system 1000 may also include a transmitter (not shown) coupled to the processor 1004 and the antenna 1018 for transmitting signals back to the telemetry unit 1006 or another unit capable of receiving the signals. For example, the electrical stimulation system 1000 may transmit signals indicating whether the electrical stimulation system 1000 is operating properly or not or indicating when the battery needs to be charged or the level of charge remaining in the battery. The processor 1004 may also be capable of transmitting information about the pulse characteristics so that a user or clinician can determine or verify the characteristics.

The above explanations are merely provided for exemplary purposes and are not intended as limiting. Since many embodiments of the invention can be made without departing from the spirit and scope of the invention, the invention also resides in the claims hereinafter appended.

What is claimed as new and desired to be protected by Letters Patent of the United States is:

1. A lead anchor, comprising:
a body having an outer surface, a top end, a from side, a first end, and a second end opposite to the first end, the body defining:
a longitudinal lead lumen extending from the first end of the body to the second end of the body said configured and arranged to receive a lead, and
a transverse lumen extending from the top end of the body and perpendicularly intersecting the lead lumen; and
a fastener disposed in the transverse lumen and configured and arranged for fastening the received lead to the lead anchor by engaging a portion of the lead, the fastener comprising a head that defines an indented pattern that is configured and arranged to receive a tip portion of a tool so that the tool can be used to rotate the fastener within the transverse lumen, wherein the indented pattern is tilted with respect to the head so that the tip portion of the tool can be inserted into the indented pattern at an angle with respect to the transverse lumen, wherein the angle is in a range from 20 to 85 degrees as measured between a longitudinal axis of the tool and a plane that contains the longitudinal axis of the lead lumen and is perpendicular to the transverse lumen.

2. The lead anchor of claim 1, further comprising at least one tab extending from the lead anchor to attach the lead anchor to patient tissue, wherein the at least one tab extends perpendicular to the transverse lumen.

3. The lead anchor of claim 2, where the at least one tab comprises a first tab and a second tab, the second tab extending in a direction opposite to the first tab, wherein the first and second tabs extend perpendicular to the transverse lumen.

4. The lead anchor of claim 1, wherein the indented pattern of the head of the fastener is configured and arranged to receive the tip portion of a ball end hex wrench tool.

5. The lead anchor of claim 1, further comprising an exterior member disposed around at least a portion of the body, the exterior member being formed of a biocompatible, flexible material.

6. The lead anchor of claim 1, further comprising a first strain relief arrangement extending along a portion of the body adjacent the first end of the body, the first strain relief portion comprising an alternating series of concentric elements with differing outer diameters, wherein the concentric elements facilitate axial deformation of the first end of the body.

7. A kit, comprising:
an implantable stimulation lead; and
the lead anchor of claim 1 configured and arranged to receive a portion of the implantable stimulation lead within the lead lumen of the lead anchor.

8. A lead anchor comprising:
a body having an outer surface, a front side, a first end, and a second end opposite to the first end, the body defining
a longitudinal lead lumen extending from the first end of the body to the second end of the body and configured and arranged to receive a lead,
a first transverse lumen extending from the outer surface of the body and perpendicularly intersecting the lead lumen, and
a second transverse lumen extending from the outer surface of the body and perpendicularly intersecting the lead lumen, wherein the first and second transverse lumen form an angle therebetween, wherein the angle is in a range from 30 to 165 degrees;
a first fastener disposed in the first transverse lumen; and
a second fastener disposed in the second transverse lumen, wherein the first and second fasteners are configured and arranged for fastening the received lead to the lead anchor by engaging a portion of the lead.

9. The lead anchor of claim 8, further comprising at least one tab extending from the lead anchor to attach the lead anchor to patient tissue.

10. The lead anchor of claim 9, where the at least one tab comprises a first tab and a second tab, the second tab extending in a direction opposite to the first tab.

11. The lead anchor of claim 8, wherein the angle is in a range from 75 to 100 degrees.

12. The lead anchor of claim 8, wherein the angle is in a range from 110 to 130 degrees.

13. The lead anchor of claim 8, further comprising a first strain relief arrangement extending along a portion of the body adjacent the first end of the body, the first strain relief portion comprising an alternating series of concentric elements with differing outer diameters, wherein the concentric elements facilitate axial deformation office first end of the body.

14. A kit, comprising:
an implantable stimulation lead; and
the lead anchor of claim 8 configured and arranged to receive a portion of the implantable stimulation lead within the lead lumen of the lead anchor.

15. A lead anchor comprising:
a body having an outer surface, a top end, a front side, a first end, and a second end opposite to the first end, the body defining
a lead lumen configured and arranged to receive a lead, the lead lumen defining an elongated opening extending along the front side of the body from the first end to the second end,
a transverse lumen extending from the top end of the body and intersecting the lead slot,
a first strain relief arrangement extending along a portion of the body adjacent the first end of the body, the first strain relief portion comprising an alternating series of first concentric elements with differing outer diameter, wherein the first concentric elements facilitate axial deformation of the first end of the body, and
a second strain relief arrangement extending along a portion of the body adjacent the second end of the body, the second strain relief portion comprising an alternating series of second concentric elements with differing outer diameters, wherein the second concentric elements facilitate axial deformation of the second end of the body; and
a fastener disposed in the transverse lumen, the fastener configured and arranged for fastening the received lead to the lead anchor by engaging a portion of the lead.

16. The lead anchor of claim 15, wherein the alternating series of first concentric elements of the first strain relief arrangement has an accordion-like structure.

17. The lead anchor of claim 15, wherein the alternating series of first concentric elements of the first strain relief arrangement is configured and arranged so that, for each first concentric element, either a) all immediately adjacent first concentric elements have a larger outer diameter or b) all immediately adjacent first concentric elements have a smaller outer diameter.

18. The lead anchor of claim 15, wherein the first concentric elements of the alternating series of first concentric elements axe concentric rings.

19. The lead anchor of claim 15, further comprising an exterior member disposed around at least a portion of the body, wherein the exterior member, the first strain relief arrangement, and the second strain relief arrangement are formed of a biocompatible, flexible material.

20. A kit, comprising:
an implantable stimulation lead; and
the lead anchor of claim 15 configured and arranged to receive a portion of the implantable stimulation lead within the lead lumen of the lead anchor.
